(19) United States
(12) Patent Application Publication
(10) Pub. No.: US 2010/0216831 A1
(43) Pub. Date: Aug. 26, 2010

(54) DESLORATADINE CRYSTALLINE FORMS MIXTURES HAVING A LOW LEVEL OF RESIDUAL SOLVENTS

(75) Inventors: Zoltan Toth, Debrecen (HU); Piroska Kovacs, Debrecen (HU); Csaba Peto, Debrecen (HU); Adrienne Kovacsne Mezei, Debrecen (HU)

Correspondence Address: MERCHANT & GOULD PC, P.O. BOX 2903, MINNEAPOLIS, MN, 55402-0903, US

(73) Assignee: Teva Pharmaceutical Industries, Ltd., Petah-Tiqva (IL)

(21) Appl. No.: 11/917,839

(22) PCT Filed: Nov. 17, 2006
(86) PCT No.: PCT/IB06/04266

§ 371 Date: Dec. 20, 2007

Related U.S. Application Data
(60) Provisional application No. 60/737,964, filed on Nov. 17, 2005.

(51) Int. Cl. A61K 31/4545; C07D 401/02

(52) U.S. Cl. 514/290; 546/93

(57) ABSTRACT

Provided are desloratadine mixtures, comprising a low level of residual solvents and processes for the preparation thereof.

This application is being filed on 17 Nov. 2006 as a PCT International Patent application in the name of Teva Pharmaceutical Industries, Ltd. an Israeli national corporation, applicant for all countries except the US, and Zoltan Toth, Piroska Kovacs, Csaba Peto, and Adrienne Kovacsne-Mezei, all citizens of Hungary, applicants for the designation of the US only. This application claims priority to U.S. Provisional Application No. 60/737,964, filed on Nov. 17, 2005, and is hereby incorporated by reference in its entirety.

### FIELD OF THE INVENTION

The present invention relates to mixtures of desloratadine Forms I and II having a low level of residual solvents, and preparation thereof.

### BACKGROUND OF THE INVENTION

Desloratadine, known as 8-chloro-6,11-dihydro-11-(4-piperidylidene)-5H-benzo[5,6]cycloheptal[1,2-b]pyridine, has the following structure:

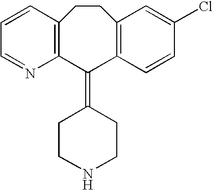

and is disclosed in U.S. Pat. No. 4,659,716. Desloratadine is currently marketed as Clarinex® in the United States. Clarinex® is prescribed as an antihistamine for prevention or treatment of allergenic reactions, which may result in symptoms such as sneezing, itchy eyes and hives. The '716 patent discloses methods for preparing and administering desloratadine and its pharmaceutically acceptable salts, and is incorporated herein by reference. See also U.S. Pat. No. 4,282,233, incorporated herein by reference, which discloses loratadine.

An aspect of the present invention relates to the solid state physical properties of desloratadine. These properties can be influenced by controlling the conditions under which desloratadine is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take this fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C-NMR spectrometry and infrared spectrometry.

The '716 patent prepares desloratadine in the solid state and discloses in Example V at column 18, lines 3-7 to extract “the organic material with chloroform, wash with water and remove the solvent. Triturate the residue with hexane. Recrystallize from a large volume of hexane after charcoal decolorization to obtain the product, m.p. 151°-152° C.”

The '716 patent prepares desloratadine in the solid state and discloses in Example VI-B at column 18, lines 43-49 that the “material is extracted several times with chloroform, the chloroform extracts washed with water and concentrated to dryness, and the residue triturated with petroleum ether or hexane to yield 11.5 grams (93%) m.p. 149°-151° C. After recrystallization from hexane, the product melts at 150°-151° C.” The starting material for Example VI-B, is an N-cyano compound prepared according to the disclosure in U.S. Pat. No. 3,326,924.

Both U.S. Pat. No. 4,282,233 and U.S. Pat. No. 3,326,924 are incorporated herein by reference, particularly for their disclosure of preparation of desloratadine.

U.S. Pat. No. 6,506,767 discloses two polymorphic forms of desloratadine, labeled Forms I and II (syn. form 1 and form 2). The XRPD peaks and the FUR spectrum for the forms are also disclosed in the '767 patent.

The '767 patent discloses at column 4, lines 21-41: “Surprisingly we discovered that certain alcoholic solvents, e.g., hexanol and methanol produced 100% polymorph form 1, but others, e.g., 3-methyl-1-butanol and cyclohexanol produced significant amounts of form 2. Chlorinated solvents, e.g., dichloromethane produced form 1 substantially free of form 2 but the compounds were discolored. Ether solvents such as dioxane produced form 1 substantially free of form 2 but other alkane ethers, e.g., di-isopropyl ether produced form 1 with significant amounts of form 2 and di-n-butyl ether favored formation of form 2. Ketones such as methyl isobutyl ketone produced crystalline polymorph form 1 essentially free of form 2 but methyl butyl ketone produced a 8:1 ratio of form 1 to form 2. Use of methyl isobutyl ketone is preferred to produce crystalline polymorph form 1 essentially free of form 2. Only ethyl acetate and di-n-butyl ether were found to produce crystalline polymorph form 2 substantially free of form 1. Use of di-n-butyl ether is preferred for producing crystalline form 2 substantially free of form 1.”

The '767 patent, in Examples 1-3, discloses the preparation of Form I by crystallization from methyl isobutyl ketone, while in examples 4 and 5, the '767 patent also discloses the preparation of Form II by crystallization from ethyl acetate and di-n-butyl ether, respectively.

The '767 patent further discloses stability tests on Polymorph Form I. According to the '767 patent at column 12, lines 12-20, Form I was “subjected to stability testing at various temperatures (25, 30 and 40° C.) and relative humidities of 60%, 60% and 75%, respectively . . . . No significant change (<1%) from initial sample % form 1 and related compounds was observed.”

The '767 patent warns against using polymorphic mixtures of desloratadine for formulation. According to the '767 patent at column 4, lines 6-11 such “a mixture could lead to [the] production of a [desloratadine] which would exist as a variable mixture of variable composition (i.e., variable percent amounts of polymorphs) having variable physical properties, a situation unacceptable in view of stringent GMP requirements.”

The '767 patent is incorporated herein by reference in its entirety, and more particularly with respect to its characterization of the polymorphic forms, synthesis of the starting material and preparation of the various polymorphic forms.

There is a need in the art for additional processes for preparation of polymorphic forms of desloratadine and pharmaceutical compositions of desloratadine.

### SUMMARY OF THE INVENTION

In one embodiment, the present invention provides a mixture of crystalline Form I and Form II of desloratadine containing about 50 ppm to about 4000 ppm of any one of isobutyl acetate, n-heptane, n-hexane, ethyl acetate, butanol, isobutanol, toluene, chloroform and combinations thereof.

In another embodiment, the present invention provides a process for preparing a mixture of crystalline Form I and Form II of desloratadine comprising combining a solution of desloratadine in an organic solvent selected from the group consisting of: a C₂ to C₅ alkyl acetate, C₁ to C₄ alcohol, C₆ to C₁₂ aromatic hydrocarbon and C₁ to C₂ chlorohydrocarbon with an anti-solvent selected from the group consisting of a C₅ to C₁₂ aliphatic hydrocarbon and C₁-C₆ symmetric or asymmetric ether to obtain a precipitate; and isolating.

In yet another embodiment, the present invention also encompasses pharmaceutical formulations comprising the mixture of crystalline Form I and Form II of desloratadine of the present invention, and pharmaceutically acceptable excipient.

In one embodiment, the present invention further encompasses a process for preparing a pharmaceutical formulation comprising combining the mixture of crystalline Form I and Form II of desloratadine of the present invention with at least one pharmaceutically acceptable excipient.

In another embodiment, the present invention further encompasses the use of the mixture of crystalline Form I and Form II of desloratadine of the present invention for the manufacture of a pharmaceutical composition.

### DETAILED DESCRIPTION OF THE INVENTION

As used herein, the term “drying” refers to removal of solvent from a solid through application of heat.

As used herein, the term “C₅ to C₁₂ saturated hydrocarbon” refers to a straight/branched and/or cyclic/acyclic hydrocarbon. Preferred hydrocarbons are cyclopentane, cyclohexane, cycloheptane, pentane, n-hexane, and n-heptane, with n-hexane and n-heptane being preferred. The terms “hexane” and “heptane” used hereinafter refer to n-hexane and n-heptane.

As used herein, the term “C₆ to C₁₂ aromatic hydrocarbon” refers to substituted and unsubstituted hydrocarbons having a phenyl group as their backbone. Preferred hydrocarbons include benzene, xylene and toluene, with toluene being more preferred.

As used herein, the term “C₃ to C₇ ester” refers to an ester having such number of carbons, such as ethyl acetate, butyl acetate, isobutyl acetate, isopropyl acetate, propyl acetate and t-butyl acetate, preferred esters include butyl acetate, isobutyl acetate, and t-butyl acetate, and more preferred esters include butyl acetate, isobutyl acetate, while most preferred esters include isobutyl acetate.

As used herein, an “anti-solvent” is a liquid that when combined with a composition comprising a solvent and desloratadine, induces precipitation of crystalline desloratadine

The crystalline desloratadine comprises a mixture of Form I and Form II. The amount of Form I and Form II is expressed herein as a weight ratio relative to each other,

$$Form I \text{-} to \text{-} Form II weight ratio = ( \frac{Form I}{Form I + Form II} ) \times 100$$

Where Form I and Form II, in the formula above, represents the amount of Form I and Form II as determined by XRPD or FTIR (see below). Likewise, the Form II-to-Form I weight ratio can be calculated by replacing the amount of Form I, in the numerator above, by the amount of Form II.

The present invention provides a mixture of crystalline Form I and Form II of desloratadine containing about 50 ppm to about 4000 ppm of any one of isobutyl acetate, n-heptane, n-hexane, ethyl acetate, butanol, isobutanol, toluene, chloroform and combinations thereof. Preferably, the mixture comprises about 35-82% desloratadine Form I and about 18-65% desloratadine Form II, more preferably, about 55-82% desloratadine Form I and 18-45% desloratadine Form II.

The present invention provides a process for preparing a mixture of crystalline Form I and Form II of desloratadine comprising combining a solution of desloratadine in an organic solvent selected from the group consisting of: a C₂ to C₅ alkyl acetate, C₁ to C₄ alcohol, C₆ to C₁₂ aromatic hydrocarbon and C₁ to C₂ chlorohydrocarbon with an anti-solvent selected from the group consisting of: a C₅ to C₁₂ aliphatic hydrocarbon and C₁-C₆ symmetric or asymmetric ether to obtain a precipitate; and isolating.

Preferably, the C₁ to C₄ alcohol is selected from the group consisting of butanol, isobutanol. Preferably, the C₆ to C₁₂ aromatic hydrocarbon is toluene. Preferably, the C₁ to C₂ chlorohydrocarbon is chloroform. Preferably, the C₂ to C₅ alkyl acetate is isobutyl acetate. Most preferably, the organic solvent is isobutyl acetate.

Preferably, the C₅ to C₁₂ aliphatic hydrocarbon is selected from the group consisting of: n-hexane, n-heptane, and combination thereof. More preferably, the C₅ to C₁₂ aliphatic hydrocarbon is n-heptane.

Preferably, the C₁-C₆ symmetric or asymmetric ether is selected from the group consisting of methyl ether, ethyl ether, propyl ether, isopropyl ether, butyl ether, 1-butyl ether, 2-butyl ether and t-butyl ether. Preferred ethers are methyl-t-butyl ether (MTBE), di-isopropyl ether and methyl ethyl ether, with di-isopropyl ether being more preferred.

When the solvent is chloroform or isobutyl acetate, examples of suitable anti-solvents include C₅ to C₁₂ aliphatic hydrocarbons, preferably saturated aliphatic hydrocarbons such as n-hexane and/or n-heptane, with n-heptane being more preferred. When it is desired to precipitate crystalline desloratadine from the composition comprising desloratadine and chloroform or isobutyl acetate, then the weight ratio of the C₅ to C₁₂ saturated hydrocarbons or ether to the chloroform or isobutyl acetate ranges from about 5% to about 500%, preferably from about 50% to about 450%, and more preferably from about 100% to about 400%, and most preferably from about 150% to about 350%, where the weight ratio of the C₅ to C₁₂ saturated hydrocarbons or ether to the chloroform or isobutyl acetate is expressed as the mass of the C₅ to C₁₂ saturated hydrocarbons or the mass of the ether to the mass per 100 g of chloroform or isobutyl acetate times 100.

Preferably, the temperature of the solution, prior to combining it with the anti-solvent, is at least about 40° C. higher than a temperature of the anti-solvent. Preferably, the temperature of the solution ranges from about 60° C. to about 100° C. and the temperature of the anti-solvent ranges from about −10° to about 40° C., prior to the combining of solution and the anti-solvent. More preferably, the temperature of the solution is about 90° C. and the temperature of the anti-solvent is about 0°, prior to the combining of the solution and the anti-solvent. Preferably, the solution is added to the anti-solvent. Preferably, the addition is carried out in less than about 10 minutes to even better retain the desired range. The difference between the temperature of the solvent and anti-solvent maintains the percentage of each crystalline form within the desired range, so that an equilibrium between the 2 forms is not obtained.

Preferably, the isolation is by filtration. Preferably, the precipitate is further dried. Preferably, the drying is by heating the mixture of crystalline Form I and Form II of desloratadine at a temperature that ranges from about 30° C. to about 100° C., more, at a temperature of from about 40° C. to about 70° C., most preferably, at a temperature of about 40° C.

Preferably, the obtained mixture of Form I and Form II comprises about 35-82% desloratadine Form I and about 18-65% desloratadine Form II. More preferably, the mixture is of about 55-82% desloratadine Form I and 18-45% desloratadine Form II.

Preferably, the processes of the present invention yield a mixture of crystalline Form I and Form II with a low level of residual solvent or anti-solvent. The amount of solvents in the product is preferably less than about 5000 ppm of any of the solvent and the anti-solvent, more preferably about 50 ppm to about 4000 ppm. Preferably, the mixture of crystalline Form I and Form II of desloratadine contains about 50 ppm to about 4000 ppm of any one of isobutyl acetate, n-heptane, n-hexane, ethyl acetate, butanol, isobutanol, toluene, chloroform and combinations thereof. Such low levels of residual solvent contribute to the solid-state stability of the mixture.

In any one of the processes of the present invention, the mixtures substantially free of solvents can be obtained on an industrial scale. Industrial batch-sizes of the present invention is preferably at least about 0.5 kg, more preferably at least about 1 kg and most preferably at least about 10 kg.

When using any one of the disclosed processes for industrial purposes, it may be advantageous to start with a desloratadine salt as a starting material since the salt is easy to handle. The desloratadine free base may then be obtained from the salt, such as the acetate salt. Desloratadine salt may be obtained by any process known in the art, such as the processes described in U.S. Pat. No. 4,659,716 and in PCT publication no. WO 2004/080461 (see also US 2004/0229896; US 2004/0242619; US 2006/0135547; and US 2006/0223841, each of which is incorporated by reference).

Desloratadine free base may be obtained by combining a desloratadine salt, such as desloratadine acetate, with an organic solvent, followed by addition of an aqueous base, at a temperature of about 50° C. to about 70° to obtain a two-phase system wherein the organic phase contains the desloratadine; separating the organic phase from the aqueous phase; and recovering desloratadine from the organic phase. Preferably, the temperature when adding the aqueous base is of about 60° C. Preferably, the obtained desloratadine free base contain minimum amount of a residual salt. Preferably, the solvent is selected from the group consisting of isobutyl acetate, butanol, isobutanol, toluene and chloroform. Most preferably, the solvent is isobutyl acetate. The base has a cationic part and an anionic part. Preferably, the cationic part is selected from the group consisting of: an alkali metal, an alkaline earth metal, a tetraalkylammonium, and combinations thereof; and the anionic part is selected from the group consisting of: an oxide, a hydroxide, a carbonate, a hydrogencarbonate, a phosphate, a hydrogenphosphate, a bis(hydrogen)phosphate, and combinations thereof. Preferably, a solution of about 2% to about 6% of sodium or potassium hydroxide, preferably about a 4% solution is used. After addition of the base, the organic phase containing desloratadine is then recovered. The organic phase may be purified by filtration. The filtrate is then preferably concentrated, more preferably by evaporation under reduced pressure. The concentration is preferably of about 5 m/m % to about 40 m/m %, more preferably about 15 m/m %.

The present invention also encompasses pharmaceutical formulations comprising the mixture of crystalline Form I and Form II of desloratadine of the present invention, and pharmaceutically acceptable excipient.

The present invention further encompasses a process for preparing a pharmaceutical formulation comprising combining the mixture of crystalline Form I and Form II of desloratadine of the present invention with at least one pharmaceutically acceptable excipient.

The present invention further encompasses the use of the mixture of crystalline Form I and Form II of desloratadine of the present invention for the manufacture of a pharmaceutical composition.

Pharmaceutical compositions of the present invention contain mixture of crystalline Form I and Form II of desloratadine. The desloratadine prepared by the processes of the present invention are ideal for pharmaceutical composition. In addition to the active ingredient(s), the pharmaceutical compositions of the present invention may contain one or more excipients.

Excipients are added to the composition for a variety of purposes. Diluents increase the bulk of a solid pharmaceutical composition, and may make a pharmaceutical dosage form containing the composition easier for the patent and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, desloratadine and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid, bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxyl toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosage's may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting including microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Capsules, tables and lozenges, and other unit dosage forms preferably contain from about 2 mg to about 20 mg of desloratadine, more preferably about 2 mg to about 10 mg of desloratadine, and most preferably about 5 mg.

Having generally described this invention, a further understanding can be obtained by reference to certain specific examples which are provided herein for purposes of illustration only and are not intended to be limiting unless otherwise specified.

### EXAMPLES

### Example 1

### Preparation of Desloratadine Mixture Form I and Form II

Desloratadine acetate (400 g) was stirred in isobutyl acetate (3000 mL) at 60° C. with 5% aqueous solution of NaOH (1000 mL, 1.2 eq). When the solid material was dissolved, the aqueous phase was removed and the organic layer was washed with distilled water (1200 mL). After phase separation the organic phase was filtered at 60° C. and the filtrate was concentrated in vacuum at 95±5° C.

A 10 mL sample of the solution is evaporated to dryness in order to check the concentration of the solution. An additional amount of isobutyl acetate (the exact amount is calculated from the determined actual concentration) was added to the solution or some of the solvent was evaporated in order to reach 16.0 m/m %. The temperature of the solution was maintained at 95±5° C.

The hot solution was added to 10000 mL n-heptane (temperature 0±5° C., stirring: 250-300 rpm) in a period of 2.0-5.5 min. The suspension was stirred for an additional ˜10 min then filtered. The wet solid was dried at 40° C. in vacuum, where one can use a slight stream of air or nitrogen in order to continuously remove the vapors from the oven. Drying time: 10 h. Residual solvents: isobutyl acetate (2700 ppm) and n-heptane (3750 ppm).

### Example 2

### Analytical HS GC Method for Determination of Residual Solvents of Desloratadine

The amount of residual solvent present in the crystalline mixture of desloratadine can be determined by the following method.

Prepare a standard stock solution by transferring about 10 mL DMSO into a 25.0 mL volumetric flask, then accurately weigh into this flask about 87.5 mg of n-Heptane, 50.0 mg of Ethanol, 100.0 mg of i-Butyl acetate, 22.0 mg of Toluene and 12.5 mg i-Butanol, fill to the mark with DMSO and homogenize. From this standard stock solution, prepare a standard solution by transferring 1.0 mL of standard stock solution into a 10.0 mL volumetric flask, fill it up with DMSO to volume and homogenize, then pipette 1.0 mL of this solution into a 20.0 mL headspace vial then close it. The resultant concentrations of the solvents are as follows: heptane: ≈350 μg/mL, ethanol: ≈200 μg/mL, isobutyl acetate: ≈400 μg/mL, toluene: ≈88 μg/mL, and isobutanol: ≈50 μg/mL). A calibration curve can be constructed in this manner so that the amount of residual solvent in the desloratadine mixture can be accurately determined comparing the results of the chromatograph with the calibration chromatograph (or curve). A typical residual solvent determination is done by accurately weighing 100.0 mg of desloratadine in a 20.0 mL headspace vial, and dissolving the weighed desloratadine with 1.0 mL DMSO with the vial closed.

Inject an appropriate quantity of the desloratadine/DMSO sample solution onto a GC column employing the following GC and headspace parameters.

### GC Parameters (Agilent 6890 N)

| Column: | Restek Stabilwax 10609 | |
|---|---|---|
| | (30 m * 0.32 mm I.D., 0.1 μm film) | |
| Carrier gas: | Helium | |
| Detector: | FID | |
| Head pressure (constant): | 100.0 | kPa |
| Nominal initial flow: | 3.4 | ml/min |
| Split ratio: | 10:1 | |
| Inlet temperature: | 140° | C. |
| Detector temperature: | 250° | C. |

Temperature program:

| | | |
|---|---|---|
| Level 1: Initial temperature: | 50° | C. |
| Initial time: | 2.0 | min |
| Level 2: Rate: | 20° | C./min |
| Final temperature: | 70° | C. |
| Final time: | 0.0 | min |
| Level 3: Rate: | 50° | C./min |
| Final temperature: | 200° | C. |
| Final time: | 0.4 | min |

Typical retention times:

| | | |
|---|---|---|
| n-Heptane and homologues | 1.3 | min |
| Ethanol | 1.6 | min |
| i-Butyl acetate | 2.0 | min |
| Toluene | 2.1 | min |
| i-Butanol | 2.4 | min |

### Headspace Parameters (Agilent 7694)

| Matrix: | Dimethyl Sulfoxide (DMSO) | | |
|---|---|---|---|
| Temperature | Sample oven: | 80° | C. |
| values: | Sample valve: | 100° | C. |
| | Transfer line: | 110° | C. |
| Time values: | GC cycle time: | 13.0 | min |
| | Sample equilibration time: | 15.0 | min |
| | Vial pressurization: | 0.20 | min |
| | Loop fill: | 0.10 | min |
| | Loop equilibration time: | 0.10 | min |
| | Inject of sample: | 0.10 | min |
| Shaking agitation: | | Low | |
| Loop volume: | | 1.0 | mL |
| Vial pressure: | | 30.0 | kPa |

A typical chromatogram is as follows (std) in which the elution times are as follows: heptane and analogues (1.3 min); ethanol (1.6 min); isobutyl acetate (2.0 min); toluene (2.1 min); and isobutanol (2.4 min).

Because toluene and isobutyl acetate have elution times that are relatively close, it may be necessary to effect dilution of the stock or sample solutions. It is determined that suitable system requirements provide for a peak resolution (Rs) standard of ≧2.5 between the i-Butyl acetate and the Toluene peaks in the chromatogram.

### Evaluation

The concentration of the residual solvent in the sample solution can be calculated from the GC data by employing the following equation.

$$Solvent content [ \% ] = \frac{{A}_{sample} * {c}_{std} * 100}{{c}_{sample} * {A}_{std}} ,$$

where: Aₛₐₘₚₗₑ is the area of the relevant solvent in the sample chromatogram; c_{std}, is the concentration of the relevant solvent in the standard solution [mg/mL]; cₛₐₘₚₗₑ, is the concentration of desloratadine in the sample solution [mg/mL]; and A_{std}, is the area of the relevant solvent in the standard chromatogram.

Having thus described the invention with reference to particular preferred embodiments and illustrative examples, those in the art would appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications, such as “Polymorphism in Pharmaceutical Solids, Drugs and the Pharmaceutical Sciences,” by Harry G. Brittain, volume 95, CRC Press, 1999, may be used as a guidance. All references mentioned herein are incorporated in their entirety.

1. A mixture of crystalline Form I and Form II of desloratadine containing about 50 ppm to about 4000 ppm of any one of isobutyl acetate, n-heptane, n-hexane, ethyl acetate, butanol, isobutanol, toluene, chloroform and combinations thereof.

2. The mixture of claim 1, wherein the mixture comprises about 35-82% desloratadine Form I and about 18-65% desloratadine Form II.

3. A process for preparing a mixture of crystalline Form I and Form II of desloratadine comprising
combining a solution of desloratadine in an organic solvent selected from the group consisting of: a C₂ to C₅ alkyl acetate, C₁ to C₄ alcohol, C₆ to C₁₂ aromatic hydrocarbon and C₁ to C₂ chlorohydrocarbon with an anti-solvent selected from the group consisting of: a C₅ to C₁₂ aliphatic hydrocarbon and C₁-C₆ symmetric or asymmetric ether to obtain a precipitate; and
isolating.

4. The process of claim 3, wherein the C₁ to C₄ alcohol is selected from the group consisting of butanol, isobutanol.

5. The process of claim 3, wherein the C₆ to C₁₂ aromatic hydrocarbon is toluene.

6. The process of claim 3, wherein the C₁ to C₂ chlorohydrocarbon is chloroform.

7. The process of claim 3, wherein the C₂ to C₅ alkyl acetate is isobutyl acetate.

8. The process of claim 3, wherein the C₅ to C₁₂ aliphatic hydrocarbon is selected from the group consisting of: n-hexane, n-heptane, and combination thereof.

9. The process of claim 3, wherein the C₁-C₆ symmetric or asymmetric ether is selected from the group consisting of methyl ether, ethyl ether, propyl ether, isopropyl ether, butyl ether, 1-butyl ether, 2-butyl ether and t-butyl ether.

10. The process of claim 3, wherein when the solvent is chloroform or isobutyl acetate, the anti-solvent is C₅ to C₁₂ aliphatic hydrocarbon.

11. The process of claim 3, wherein the temperature of the solution, prior to combining it with the anti-solvent, is at least about 40° C. higher than a temperature of the anti-solvent.

12. The process of claim 11, wherein the temperature of the solution, prior to combining it with the anti-solvent, ranges from about 60° C. to about 100° C. and the temperature of the anti-solvent ranges from about −10° to about 40° C.

13. The process of claim 12, wherein the temperature of the solution, prior to combining it with the anti-solvent, is about 90° C. and the temperature of the anti-solvent is about 0° C.

14. The process of claim 3, wherein the solution is added to the anti-solvent.

15. The process of claim 14, wherein the addition is carried out in less than about 10 minutes.

16. The process of claim 3, wherein the isolation is by filtration.

17. The process of claim 3, wherein the process further comprises drying the precipitate.

18. The process of claim 17, wherein the drying is by heating the mixture of crystalline Form I and Form II of desloratadine at a temperature that ranges from about 30° C. to about 100° C.

19. The process of claim 18, wherein the drying is by heating the mixture of crystalline Form I and Form II of desloratadine at a temperature that ranges from about 40° C. to about 70° C.

20. The process of claim 3, wherein the obtained mixture of Form I and Form II comprises about 35-82% desloratadine Form I and about 18-65% desloratadine Form II.

21. The process of claim 20, wherein the obtained mixture of Form I and Form II comprises about 55-82% desloratadine Form I and 18-45% desloratadine Form II.

22. The process of claim 3, wherein the mixture of crystalline Form I and Form II contains less than about 5000 ppm of any of the solvent and the anti-solvent.

23. The process of claim 22, wherein the mixture of crystalline Form I and Form II contains about 50 ppm to about 4000 ppm of any of the solvent and the anti-solvent.

24. The process of claim 23, wherein the mixture of crystalline Form I and Form II of desloratadine contains about 50 ppm to about 4000 ppm of any one of isobutyl acetate, n-heptane, n-hexane, ethyl acetate, butanol, isobutanol, toluene, chloroform and combinations thereof.

25. Pharmaceutical formulations comprising the mixture of crystalline Form I and Form II of desloratadine of claim 1, and pharmaceutically acceptable excipient.

26. A process for preparing a pharmaceutical formulation comprising combining the mixture of crystalline Form I and Form II of desloratadine of claim 1, with at least one pharmaceutically acceptable excipient.

27. (canceled)

28. The mixture of claim 1, wherein the mixture comprises about 55-82% desloratadine Form I and about 18-55% desloratadine Form II.

29. Pharmaceutical formulations comprising the mixture of crystalline Form I and Form II of desloratadine of claim 2 and pharmaceutically acceptable excipient.

30. A process for preparing a pharmaceutical formulation comprising combining the mixture of crystalline Form I and Form II of desloratadine of claim 2, with at least one pharmaceutically acceptable excipient.
